(19) United States
(12) Patent Application Publication
(10) Pub. No.: US 2014/0186921 A1
(43) Pub. Date: Jul. 3, 2014

(54) LOW PH PROTEIN PURIFICATION PROCESS

(71) Applicants: Thomas Strömquist, Stockholm (SE); Susanne Wood, Nacka (SE)

(72) Inventors: Thomas Strömquist, Stockholm (SE); Susanne Wood, Nacka (SE)

(73) Assignee: SWEDISH ORPHAN BIOVITRUM AB (PUBL), Stockholm (SE)

(21) Appl. No.: 14/117,331

(22) PCT Filed: May 15, 2012
(86) PCT No.: PCT/SE2012/050519

§ 371 Date: Jan. 8, 2014

(30) Foreign Application Priority Data
May 18, 2011 (SE) 1150454-5

(51) Int. Cl. C12N 9/20

(52) U.S. Cl. CPC C12N 9/20
USPC 435/198

(57) ABSTRACT

The invention relates to a process for recovering and purifying bile salt-stimulated lipase (BSSL) in a solution which contains impurities, said process comprising the steps: (i) applying BSSL to a hydrophobic interaction chromatography (HIC) resin; (ii) removing impurities by washing said HIC resin with a wash composition having a pH in the range from 4 to 5; and (iii) recovering BSSL from said HIC resin.

### TECHNICAL FIELD

The invention relates to methods for purifying bile salt-stimulated lipase (BSSL), said methods comprising the use of hydrophobic interaction chromatography at low pH and, optionally, anion-exchange chromatography at low pH.

### BACKGROUND ART

The human lactating mammary gland and pancreas produce a lipolytic enzyme, bile salt-stimulated lipase (BSSL), also referred to as bile salt-activated lipase (BAL) or carboxylic ester lipase (CEL). BSSL is a major component of pancreatic juice and is responsible for the hydrolysis of cholesterol esters as well as a variety of other dietary esters. The enzyme exerts its function in duodenal juice, is activated when mixed with bile salts, and plays an important role in the digestion of milk fat in newborn infants (for a review, see e.g. Wang & Hartsuck (1993) Biochim. Biophys Acta 1166: 1-19).

BSSLs from human milk and human pancreas have been purified and characterized, as reported by Wang (1980; Anal. Biochem. 105: 398-402); Blackberg & Hernell (1981; Eur J Biochem, 116: 221-225); Wang & Johnson (1983; Anal. Biochem. 133: 457-461); Wang (1988; Biochem. Biophys. Res. Comm. 164: 1302-1309). The cDNA sequence of human BSSL was identified by Nilsson (1990; Eur J Biochem, 192: 543-550) and disclosed in WO 91/15234 and WO 91/18923.

However, it has not been previously disclosed that BSSL can be purified by methods involving hydrophobic interaction chromatography and/or anion exchange chromatography, wherein the chromatography resin is washed at low pH. There is a need for improved methods for the purification of BSSL, which methods are capable of efficiently removing impurities such as host cell proteins (HCP) and DNA, while at the same time give a high yield of product.

### BRIEF DESCRIPTION OF THE DRAWINGS

FIG. 1 shows the amount of host cell proteins (ng/mg) in products obtained after anion exchange chromatography (DEAE) by purification methods A, B and C, respectively. In FIGS. 1-6, the error bars indicate the confidence interval (95% confidence level).

FIG. 2 shows the amount of DNA (pg/mg) in products obtained after DEAE.

FIG. 3 shows the yield (%) of BSSL after hydrophobic interaction chromatography (HIC).

FIG. 4 shows the amount of host cell proteins (ng/mg) in products obtained after HIC.

FIG. 5 shows the amount of DNA (pg/mg) in products obtained after HIC.

FIG. 6 shows the yield (%) of BSSL after DEAE and HIC in combination.

FIG. 7 shows the log reduction of host cell proteins in products obtained after DEAE and HIC in combination.

FIG. 8 shows the log reduction of DNA in products obtained after DEAE and HIC in combination.

### DISCLOSURE OF THE INVENTION

It has surprisingly been found that bile salt-stimulated lipase (BSSL) can advantageously be purified by hydrophobic interaction chromatography (HIC) even at low pH. Impurities, exemplified by host cell proteins (HCP) and DNA, are efficiently removed with this method and a more pure product is obtained, while product yield is maintained. In particular, the invention provides a method hereinafter referred to as “Method A”, which is useful for the purification of BSSL. Method A comprises a combination of (a) anion-exchange chromatography, comprising washing the column at low pH and eluting BSSL at low pH; and (b) hydrophobic interaction chromatography, comprising washing the column at low pH.

Consequently, in a first aspect this invention provides a process for recovering and purifying bile salt-stimulated lipase (BSSL) in a solution which contains impurities, said process comprising the steps:
(i) applying BSSL to a hydrophobic interaction chromatography (HIC) resin;
(ii) removing impurities by washing said HIC resin with a wash composition having a pH in the range from 3.5 to 5, preferably from 3.5 to 4.5, and more preferably about pH 4; and
(iii) recovering BSSL from said HIC resin.

The term “hydrophobic interaction chromatography (HIC)” is well known in the art and refers to a separation technique that uses the properties of hydrophobicity to separate proteins from one another. In this separation, a buffer with a high ionic strength is initially applied to the column and to the sample. The salt in the buffer causes protein conformance changes and exposing of hydrophobic regions that are adsorbed to the medium. To elute the proteins, the salt concentration is decreased.

The term “impurities” refers in particular to host cell proteins and DNA from the cells used for production of the target protein and which will be present in the cultivation broth.

The said BSSL is preferably human BSSL, more preferably recombinant human BSSL. Recombinant human BSSL can be produced by methods known in the art, for instance by expression in recombinant Chinese hamster ovary (CHO) cells, as described below in the experimental section. Alternatively, recombinant BSSL can be produced in other known expression systems such as E. coli, as described by Hansson et al. (1993) J. Biol. Chem. 268: 26692-26698; or Pichia pastoris, as disclosed in WO 96/37622.

In a preferred form of the invention, the BSSL purification process comprises an anion-exchange chromatography step wherein BSSL is washed an eluted at low pH, such as pH 4-5. Consequently, the invention provides a process as described above (comprising HIC) and in addition comprising the steps:
(i) applying BSSL to an anion-exchange resin;
(ii) removing impurities by washing said anion-exchange resin with a wash composition having a pH in the range from 4 to 5, preferably from about 4.4 to about 4.6, such as pH 4.4 or 4.5; and
(iii) recovering BSSL by eluting said anion-exchange resin with an eluant. Preferably, the said eluant has a pH in the range from 4 to 5, preferably from about 4.4 to about 4.6, such as pH 4.4 or 4.5.

The term “anion-exchange chromatography” (AIEX) is well known in the art and refers to a separation technique which involves binding of negatively charged amino acids to an immobilized cation surface. Normally, biomolecules are released from the anion exchanger by changing the buffer composition, such as increasing the ionic strength with sodium chloride. It is particularly preferred that the anion-exchange step is carried out prior to the HIC step, i.e. BSSL is recovered from the anion-exchange resin prior to being applied to the HIC resin.

In a particularly preferred form of the invention, the BSSL purification process is the process referred to as “Method A” in the Examples and comprises the following steps:
(i) applying BSSL to an anion-exchange resin;
(ii) removing impurities by washing said anion-exchange resin with a wash composition having a pH in the range from 4 to 5, preferably from about 4.4 to about 4.6, such as pH 4.4 or 4.5.;
(iii) recovering BSSL by eluting said anion-exchange resin with an eluant, preferably having a pH in the range from 4 to 5, and more preferably from about 4.4 to about 4.6, such as pH 4.4 or 4.5;
(iv) applying BSSL obtained in step (iii) to a hydrophobic interaction chromatography (HIC) resin;
(v) removing impurities by washing said HIC resin with a wash composition having a pH in the range from 3.5 to 5, preferably from 3.5 to 4.5, and more preferably about pH 4, and
(vi) recovering BSSL from said HIC resin.

It will be understood by the skilled person that additional steps can be included in the purification methods according to the invention. For instance, one or more additional steps can be included in “Method A” either before the AIEX, between the AIEX and the HIC, or after the HIC. Examples of such additional steps include virus reduction steps, ultrafiltration and diafiltration (UF/DF), etc.

### EXAMPLES

### 1. Expression of Recombinant BSSL

Human BSSL can be produced by expression from recombinant Chinese hamster ovary (CHO) cells containing a nucleic acid expression system comprising the nucleotide sequence encoding human BSSL according to standard procedures. Briefly, the 2.3 Kb cDNA sequence encoding full-length hBSSL including the leader sequence (as described by Nilsson et al, 1990; Eur J Biochem, 192: 543-550) is obtained from pS146 (Hansson et al, 1993; J Biol Chem, 268: 26692-26698) and cloned into the expression vector pAD-CMV 1 (Boehringer Ingelheim)—a pBR-based plasmid that includes CMV promoter/SV40 polyA signal for gene expression and the dhfr gene for selection/amplification—to form pAD-CMV-BSSL.

pAD-CMV-BSSL is then used for transfection of DHFR-negative CHOss cells (Boehringer Ingelheim)—together with co-transfection of plasmid pBR3127 SV/Neo pA coding for neomycin resistance to select for geneticin (G418) resistance—to generate DHFR-positive BSSL producing CHO cells. The resulting CHO cells are cultured under conditions and scale to express larger quantities of rhBSSL. For example, cells from the master cell bank (MCB) are thawed, expanded in shaker flasks using Ex-Cell 302 medium without glutamine and glucose (SAFC) later supplemented with glutamine and glucose, followed by growth in 15 and 100 L bioreactors, before inoculating the 700 L production bioreactor where BSSL is constitutively expressed and produced in a fed-batch process. Harvested material from the cell cultivation can be clarified either by using a combination of depth and absolute filters, or by centrifugation.

### 2. Purification of BSSL (Method A)

### Anion-Exchange Chromatography

Clarified harvest from a CHO cell culture expressing BSSL was diluted (about 1:1.2, from 17 to 9 mS/cm) with Tris buffer (10 mM, pH 7). The diluted harvest was loaded onto a DEAE Sepharose FFTM anion exchange column (GE Healthcare). Following an initial wash (“Wash 1”) with Tris buffer (25 mM, pH 7.2), the column was washed (“Wash 2”) with a buffer comprising 25 mM sodium acetate (pH 4.5) and 50 mM sodium chloride. BSSL was step-eluted from the column with a buffer comprising 25 mM sodium acetate (pH 4.5) and 350 mM NaCl.

### Virus Inactivation

For low pH virus inactivation according to known methods, pH in the DEAE pool was decreased to 3.5 by addition of glycine-HCl, pH 2.5. After 60 min incubation, pH was increased to 6.3 by addition of 0.5 M dibasic sodium phosphate, pH 9.

### Hydrophobic Interaction Chromatography

After virus inactivation, BSSL was conditioned to a conductivity of about 140 mS/cm by addition of 4 M sodium chloride/25 mM sodium phosphate (pH 6). The final sodium chloride concentration was about 1.75 M. The sample was loaded on a Phenyl Sepharose FFTM high substitution column (GE Healthcare). The column was washed (“Wash 1”) with a buffer comprising 25 mM sodium phosphate (pH 6) and 1.75 M sodium chloride. The column was then washed (“Wash 2”) with 25 mM sodium acetate, pH 4, and 1.75 M sodium chloride. The column was finally washed (“Wash 3”) with the same buffer as in “Wash 1” (25 mM sodium phosphate, pH 6, and 1.75 M sodium chloride). BSSL was then eluted by lowering the conductivity (10 mM sodium phosphate, pH 6).

### 3. Purification of BSSL (Method B for Comparison)

BSSL was purified by “Method B” which was identical to Method A, above, except that “Wash 2” was excluded both in the anion exchange step and in the HIC step. Further, during anion exchange chromatography, BSSL was eluted at pH 7.2, using Tris buffer.

### 4. Purification of BSSL (Method C for Comparison)

BSSL was purified by “Method C” which was identical to Method A, above, except for the following steps:
(i) during anion exchange chromatography, the “Wash 2” and elution steps were carried out at pH 7.2, using Tris buffer; and
(ii) during HIC, “Wash 2” was carried out at pH 6, using sodium phosphate buffer.

Tables I and II, below, summarize the differences between methods A, B and C during anion exchange chromatography and HIC, respectively.

TABLE I
Anion exchange chromatography of BSSL
(CV = Column volumes)

| | | | | Flow |
|---|---|---|---|---|
| | | | Length | rate |
| Method | Step | Buffer | (CV) | (cm/h) |
| ALL | Equilibration | 25 mM Tris, pH 7.2 | 4 | 250 |
| | Sample | 10 mM Tris, pH 7.2 | ~35 | 250 |
| | application | | | |
| | Wash 1 | 25 mM Tris, pH 7.2 | 3 | 250 |
| A | Wash 2 | 25 mM NaAc, 50 mM NaCl, | 7 | 250 |
| | | pH 4.5 | | |
| | Elution | 25 mM NaAc, 350 mM NaCl, | 3 | 250 |
| | | pH 4.5 | | |
| B | Wash 2 | (Excluded) | — | — |
| | Elution | 25 mM Tris, 350 mM NaCl, | 3 | 250 |
| | | pH 7.2 | | |
| C | Wash 2 | 25 mM Tris, 50 mM NaCl, | 7 | 250 |
| | | pH 7.2 | | |
| | Elution | 25 mM Tris, 350 mM NaCl, | 3 | 250 |
| | | pH 7.2 | | |

TABLE II
Hydrophobic interaction chromatography of BSSL
(CV = Column volumes)

| | | | | Flow |
|---|---|---|---|---|
| | | | Length | rate |
| Method | Step | Buffer | (CV) | (cm/h) |
| ALL | Equilibration | 1.75M NaCl, 25 mM NaP, | 4 | 250 |
| | | pH 6 | | |
| | Sample | 1.75M NaCl, 25 mM NaP, | — | 250 |
| | application | pH 6 | | |
| | Wash 1 | 1.75M NaCl, 25 mM NaP, | 2 | 250 |
| | | pH 6 | | |
| A | Wash 2 | 1.75M NaCl, 25 mM NaAc, | 7 | 250 |
| | | pH 4 | | |
| B | | (Excluded) | — | — |
| C | | 1.75M NaCl, 25 mM NaP, | 7 | 250 |
| | | pH 6 | | |
| ALL | Wash 3 | 1.75M NaCl, 25 mM NaP, | 3 | 250 |
| | | pH 6 | | |
| | Elution | 10 mM NaP, pH 6 | 3 | 250 |

5. Results from Methods A-C

### Anion Exchange Chromatography

Table III shows results from purification of BSSL by anion exchange chromatography, including low-pH virus inactivation. As shown in the column “Yield” most product was recovered, as expected, with Method B in which “Wash 2” was excluded. However, Table III also shows that more product is recovered with Method A (“Wash 2” at pH 4.5) than with Method C (“Wash 2” at pH 7.2).

TABLE III
Results from anion exchange chromatography

| | | HCP | | | DNA | | | SE-HPLC |
|---|---|---|---|---|---|---|---|---|
| Method | Yield | ng/mL | ng/mg | LRV | pg/mL | pg/mg | LRV | Main Peak (%) |
| A | 73% | 195000 | 57353 | 1.0 | 1.50 · 10⁵ | 4.41 · 10⁴ | 2.1 | 84.9 |
| B | 82% | 236000 | 76129 | 0.8 | 1.80 · 10⁶ | 5.81 · 10⁵ | 0.9 | 80.6 |
| C | 67% | 186000 | 43256 | 1.1 | 2.70 · 10⁶ | 6.28 · 10⁵ | 0.9 | 82.0 |

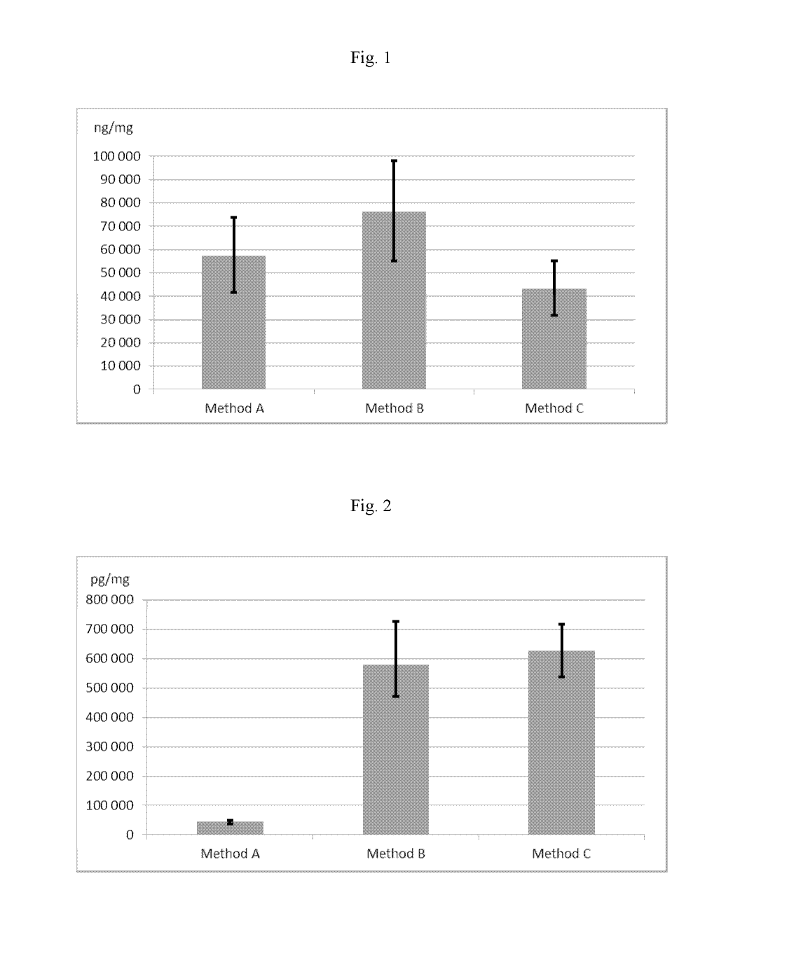

Table III and FIG. 1 show the host cell protein (HCP) content in the material obtained from anion exchange chromatography. From these data, Methods A-C appear to be similarly effective with regard to HCP removal. However, analysis on SDS-PAGE (not shown) revealed that bands, representing proteins of sizes and charges different from BSSL, were stronger in Method B and C samples, indicating that Method A provides material with less HCP.

Table III and FIG. 2 show DNA content in the material obtained from anion exchange chromatography. Surprisingly, Method A proved to clear more DNA while maintaining effectiveness of processing the product, resulting in Method A being significantly more effective than Methods B and C for clearance of DNA in the obtained product.

As further shown in Table III, analysis by SE-HPLC (Size exclusion-high performance to liquid chromatography) according to known methods indicates that a more pure product (“Main Peak”, corresponding to full-length BSSL) is obtained with Method A than with Methods B or C.

### Hydrophobic Interaction Chromatography

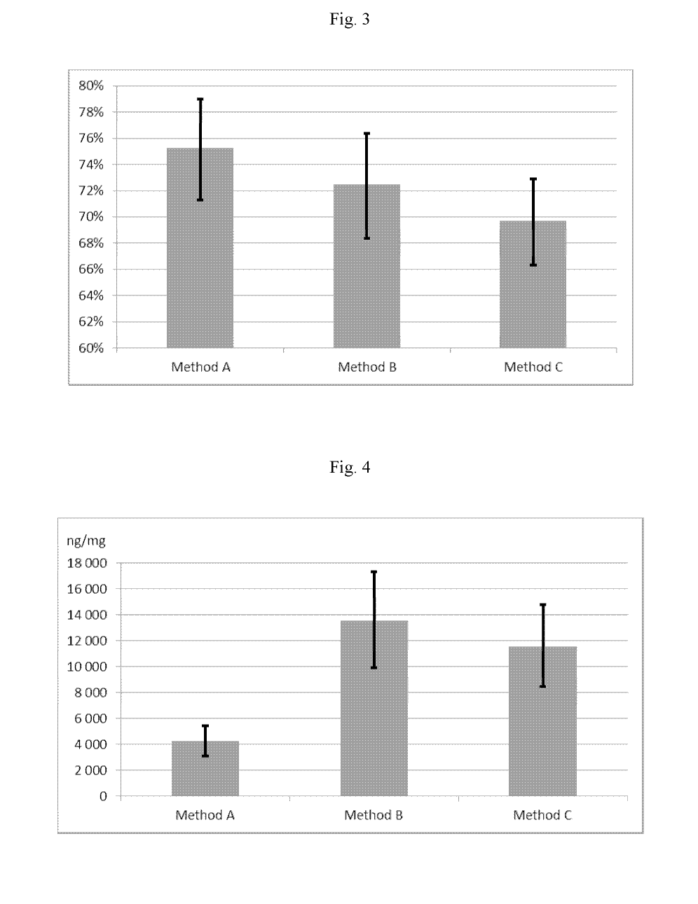

As shown in Table IV (“Yield”) and FIG. 3, the product yield was similar with all three methods. Nevertheless, Method A surprisingly achieved a slightly better product yield in comparison with Methods B and C.

TABLE IV
Results from hydrophobic interaction chromatography

| | | | | | | | | SE-HPLC | | |
|---|---|---|---|---|---|---|---|---|---|---|
| | | HCP | | | DNA | | | LMW | Monomer | HMW |
| Method | Yield | ng/mL | ng/mg | LRV | pg/mL | pg/mg | LRV | (%) | (%) | (%) |
| A | 75% | 19700 | 4283 | 1.3 | 2.70 · 10³ | 587 | 2.0 | 3.7 | 95.5 | 0.9 |
| B | 73% | 58300 | 13558 | 0.9 | 1.60 · 10⁴ | 3720 | 2.3 | 4.1 | 95.0 | 1.0 |
| C | 70% | 48500 | 11548 | 0.7 | 1.70 · 10⁴ | 4050 | 2.4 | 5.0 | 94.2 | 0.7 |

Table IV and FIG. 4 show the host cell protein (HCP) content in the material obtained from hydrophobic interaction chromatography. The data shows that Method A was superior to Methods B and C with regard to removal of HCP. The same results were obtained with SDS-PAGE (not shown).

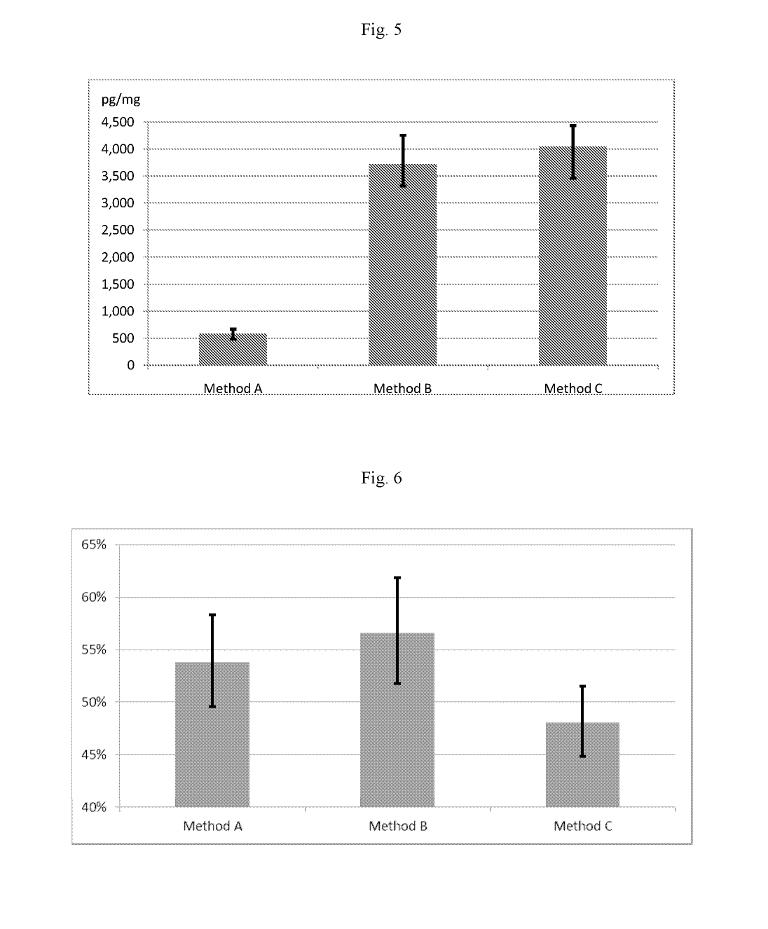

Table IV and FIG. 5 show DNA content in the material obtained from hydrophobic interaction chromatography. Again, Method A showed to be superior to Methods B and C in removing DNA from the product pool. With Methods B and C, the amount of residual DNA per amount of product is more than 6 times higher than the corresponding amount with Method A. Further, Table IV shows that according to SE-HPLC analysis, the highest amounts of monomeric BSSL, as well as least amount of low molecular weight (LMW) material, were obtained with Method A.

### Conclusions

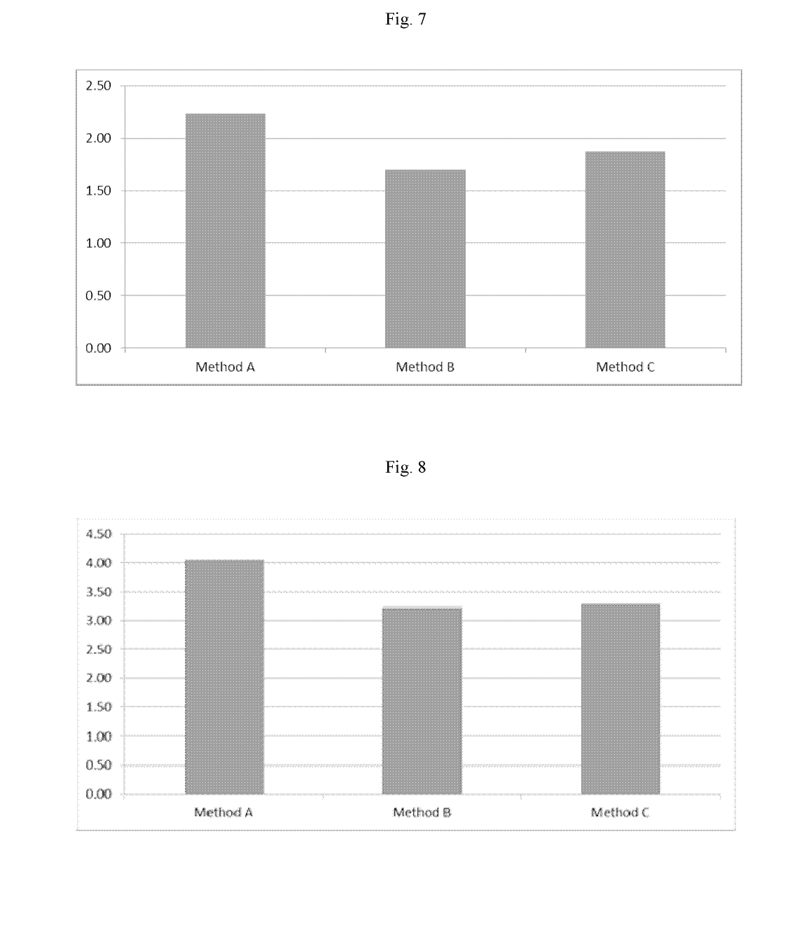

When the results from anion exchange and HIC are combined, it is shown that there was no significant difference between product yields obtained with Methods A, B and C (FIG. 6). However, log reduction values (LRV) for the contaminants HCP (FIG. 7) and DNA (FIG. 8), were superior with Method A in comparison with Methods B and C. The Log Reduction Value is the logarithm (log10) of the ratio between the total amount of impurities loaded into the step and the total amount of impurities after the step (in the intermediate pool).

In summary, “Method A” for purification of BSSL comprises a combination of (a) anion-exchange chromatography, comprising washing the column at low pH and eluting

BSSL at low pH; and (b) hydrophobic interaction chromatography, comprising washing the column at low pH. It has it has surprisingly been found that with “Method A”, impurities, exemplified by host cell proteins (HCP) and DNA, are efficiently removed and a more pure product is obtained, while product yield is maintained.

1. A process for recovering and purifying bile salt-stimulated lipase (BSSL) in a solution which contains impurities, said process comprising the steps:
(i) applying BSSL to a hydrophobic interaction chromatography (HIC) resin;
(ii) removing impurities by washing said HIC resin with a wash composition having a pH in the range from 3.5 to 5; and
(iii) recovering BSSL from said HIC resin.

2. The process according to claim 1 wherein the BSSL is human BSSL.

3. The process according to claim 2 wherein the BSSL is recombinant human BSSL.

4. The process according to claim 3 wherein the recombinant human BSSL is produced by expression from recombinant Chinese hamster ovary (CHO) cells.

5. The process according to claim 1 wherein the pH of the wash composition is from 3.5 to 4.5.

6. The process according to claim 5 wherein the pH of the wash composition is 4.

7. The process according to claim 1, said process in addition comprising the steps:
(i) applying BSSL to an anion-exchange resin;
(ii) removing impurities by washing said anion-exchange resin with a wash composition having a pH in the range from 4 to 5; and
(iii) recovering BSSL.

8. The process according to claim 7 wherein BSSL is recovered by eluting said anion-exchange resin with an eluant having a pH in the range from 4 to 5.

9. The process according to claim 7, wherein BSSL is recovered from the anion-exchange resin prior to being applied to the HIC resin.

10. The process according to claim 9, said process comprising the steps:
(i) applying BSSL to an anion-exchange resin;
(ii) removing impurities by washing said anion-exchange resin with a wash composition having a pH in the range from 4 to 5;
(iii) recovering BSSL by eluting said anion-exchange resin with an eluant having a pH in the range from 4 to 5;
(iv) applying BSSL obtained in step (iii) to a hydrophobic interaction chromatography (HIC) resin;
(v) removing impurities by washing said HIC resin with a wash composition having a pH in the range from 3.5 to 5; and
(vi) recovering BSSL from said HIC resin.

11. The process according to claim 10 wherein the wash composition in step (ii) has a pH in the range from 4.4 to 4.6.

12. The process according to claim 10 wherein the eluant in step (iii) has a pH in the range from 4.4 to 4.6.

13. The process according to claim 10 wherein pH in the wash composition in step (v) is from 3.5 to 4.5.

14. The process according to claim 13 wherein pH in the wash composition in step (v) is 4.
